(19) United States
(12) Patent Application Publication
(10) Pub. No.: US 2011/0144181 A1
(43) Pub. Date: Jun. 16, 2011

(54) Pharmaceutical Compositions of Amorphous Atorvasta and Process for Preparing Same

(75) Inventors: Stephen Craig Dyar, Brighton, MI (US); Mayur P. Lodaya, Northville, MI (US); Matthew J. Mollan, JR., Northville, MI (US); Umang Shah, Ann Arbor, MI (US); Hei Jen Sun, Ann Arbor, MI (US); Zeri Teweldemedhin, Ypsilanti, MI (US)

(73) Assignee: Warner-Lambert Company LLC

(21) Appl. No.: 13/034,230

(22) Filed: Feb. 24, 2011

Related U.S. Application Data
(63) Continuation of application No. 11/795,438, filed on Mar. 27, 2008.
(60) Provisional application No. 60/632,559, filed on Dec. 2, 2004.

(51) Int. Cl. A61K 31/40

(52) U.S. Cl. 514/423

(57) ABSTRACT

Solid pharmaceutical compositions containing atorvastatin are disclosed. The compositions include a solid dispersion of amorphous atorvastatin and one or more optional pharmaceutically acceptable excipients. The solid dispersion is prepared by mixing crystalline atorvastatin with a melt-processible polymer and an optional stabilizer and an optional plasticizer at a temperature sufficiently high to soften or melt the polymer and to melt or dissolve the crystalline atorvastatin in the polymer, thereby forming a dispersion of amorphous atorvastatin.

### BACKGROUND OF THE INVENTION

1. Field of Invention

This invention relates to methods and materials for preparing solid pharmaceutical compositions containing amorphous atorvastatin and to stable pharmaceutical compositions of amorphous atorvastatin prepared via hot melt extrusion.

2. Discussion

Atorvastatin calcium or [R-(R*,R*)]-2-(4-fluorophenyl)-β,δ-dihydroxy-5-(1-methylethyl)-3-phenyl-4-[(phenylamino)carbonyl]-1H-pyrrole-1-heptanoic acid calcium salt (2:1) trihydrate, is the active pharmaceutical ingredient in LIPITOR® and is represented by the structural formula:

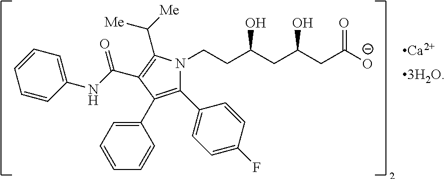

Atorvastatin and its pharmaceutically acceptable complexes, salts, solvates, and hydrates are selective, competitive inhibitors of 3-hydroxy-3-methylglutaryl-coenzyme A (HMG-CoA) reductase, which catalyzes the conversion of HMG-CoA to mevalonate—an early and rate-limiting step in the cholesterol biosynthetic pathway. See U.S. Pat. No. 5,273,995 to B. D. Roth, which is herein incorporated by reference.

When compared to a drug's crystalline form (or forms) an amorphous form (or forms) of the same drug may exhibit different in vitro dissolution characteristics. The amorphous form may also exhibit different bioavailability, which for drugs intended to provide systemic therapeutic effect, may be characterized by differences in the pharmacokinetic (PK) profile or drug plasma concentration as a function of time. See T. Konno, Chem. Pharm. Bull. 38:2003-2007 (1990). For some therapeutic indications, one PK profile may provide advantages over another. Thus, for instance, some potential uses of atorvastatin may benefit from comparatively rapid absorption of the drug into the bloodstream. See, e.g., M. Takemoto et al., Journal of Clinical Investigation 108(10):1429-1437 (2001), which describes the acute treatment of stroke patients.

Various methods for preparing amorphous atorvastatin have been described, and many of these methods employ volatile organic solvents. For example, U.S. Pat. No. 6,087,511 to Lin et al. describes forming amorphous atorvastatin by dissolving crystalline atorvastatin in a non-hydroxylic solvent, such as tetrahydrofuran, and subsequently removing the non-hydroxylic solvent to give amorphous atorvastatin. See also, WO 00/71116 to Y. Kumar et al.; WO 01/28999 to Z. Graff et al.; and WO 01/42209 to Z. Phlaum, the complete disclosures of which are herein incorporated by reference. Once amorphous atorvastatin has been prepared, it may be combined with pharmaceutically acceptable excipients to give pharmaceutical compositions and dosage forms.

What would be desirable are methods for preparing amorphous atorvastatin and stable pharmaceutical compositions containing amorphous atorvastatin, which do not involve the use of volatile organic solvents.

### SUMMARY OF THE INVENTION

The present invention provides a solid pharmaceutical composition, which comprises a solid dispersion of amorphous atorvastatin and one or more optional pharmaceutically acceptable excipients. In one embodiment, the solid dispersion includes amorphous atorvastatin or a pharmaceutically acceptable complex, salt, solvate or hydrate thereof, and a melt-processible polymer. In another embodiment, the solid dispersion includes amorphous atorvastatin or a pharmaceutically acceptable complex, salt, solvate or hydrate thereof, a melt-processible polymer, and a stabilizer for reducing chemical degradation of the amorphous atorvastatin.

A further aspect of the present invention provides a method of making a solid pharmaceutical composition. The method includes steps of: (a) mixing crystalline atorvastatin or a pharmaceutically acceptable complex, salt, solvate or hydrate thereof, with a melt-processible polymer at a temperature sufficiently high to soften or melt the melt-processible polymer and to melt or dissolve the crystalline atorvastatin in the melt-processible polymer, thereby forming a dispersion of amorphous atorvastatin; and (b) allowing the dispersion to cool.

### BRIEF DESCRIPTION OF THE DRAWINGS

FIG. 1 shows a PXRD (1.54 Å) diffractogram for crystalline atorvastatin calcium.

FIG. 2 shows a PXRD (1.54 Å) diffractogram for CaCO₃.

FIG. 3 shows PXRD (1.54 Å) diffractograms for the solid dispersions of atorvastatin in Example 1 to Example 7 following hot-melt extrusion and milling.

FIG. 4 shows PXRD (1.54 Å) patterns for the formulation in Example 6 after blending, but before extrusion (diffractogram A); following extrusion and milling, but before storage (diffractogram B); following extrusion and milling and subsequent exposure to 40° C. and 75% RH for 60 hours (diffractogram C).

FIG. 5 shows PXRD (1.54 Å) diffractograms for the solid dispersions of atorvastatin in Example 8, 10, 12, 14, and 16 following hot-melt extrusion and milling.

FIG. 6 shows PXRD (1.54 Å) diffractograms for the solid dispersions of atorvastatin in Example 9, 11, 13, 15, and 17 following hot-melt extrusion and milling.

FIG. 7 shows PXRD (1.54 Å) diffractograms for the solid dispersions of atorvastatin in Example 8, 10, 12, 14, and 16 following hot-melt extrusion, milling and subsequent exposure to 40° C. and 75% RH for 3 months.

FIG. 8 shows PXRD (1.54 Å) diffractograms for the solid dispersions of atorvastatin in Example 9, 11, 13, 15, and 17 following hot-melt extrusion, milling and subsequent exposure to 40° C. and 75% RH for 3 months.

### DETAILED DESCRIPTION

### Definitions and Abbreviations

Unless otherwise indicated, this disclosure uses definitions provided below.

“About,” “approximately,” and the like, when used in connection with a numerical variable, generally refers to the value of the variable and to all values of the variable that are within the experimental error (e.g., within the 95% confidence interval for the mean) or within ±10% of the indicated value, whichever is greater.

“Pharmaceutically acceptable” refers to substances, which are within the scope of sound medical judgment, suitable for use In contact with the tissues of patients without undue toxicity, irritation, allergic response, and the like, commensurate with a reasonable benefit/risk ratio, and effective for their intended use.

“Treating” refers to reversing, alleviating, inhibiting or slowing the progress of, or preventing a disorder or condition to which such term applies, or to preventing one or more symptoms of such disorder or condition.

“Treatment” refers to the act of “treating.”

“Drug,” “drug substance,” “active pharmaceutical ingredient,” and the like, refer to a compound that may be used for treating a patient in need of treatment.

“Excipient” or “adjuvant” refers to any component of a pharmaceutical composition that is not the drug substance.

“Drug product,” “pharmaceutical dosage form,” “final dosage form,” and the like, refer to the combination of one or more drug substances and one or more excipients (i.e., pharmaceutical composition) that is administered to a patient in need of treatment, and may be in the form of tablets, capsules, liquid suspensions, patches, and the like.

“Inert” refers to substances that may positively influence the bioavailability of the drug, but are otherwise unreactive.

“Amorphous” refers to solid-state particles that lack a regular crystalline structure and as a consequence give a diffuse, i.e., non-distinctive, powder x-ray diffraction (PXRD) pattern.

“Crystalline” refers to solid-state particles having a regular ordered structure, which, in contrast to amorphous material, give a distinctive PXRD pattern with defined peaks.

“Solid dispersion,” “amorphous solid dispersion,” and the like, refer to a drug substance, which has been dispersed or distributed in a carrier or dispersion medium. Generally, at least a portion, and in many cases a majority, of the drug substance is amorphous. The drug may be present in the dispersion as (a) discrete, drug-rich domains or may be (b) homogeneously distributed throughout the carrier (i.e., a solid solution) or may be some combination of (a) and (b). For a discussion of pharmaceutical solid dispersions, see W. L. Chiou & S. Riegelman, J. Pharm. Sci 60(9):1282-1302 (1971), which is herein incorporated by reference.

“Particle size” refers to the median or to the average dimension of particles in a sample and may be based on the number of particles, the volume of particles, or the mass of particles, and may be obtained using any number of standard measurement techniques, including laser diffraction methods, centrifugal sedimentation techniques, photon correlation spectroscopy (dynamic light scattering or quasi-elastic light scattering), or sieving analysis using standard screens. Unless stated differently, all references to particle size in this specification refer to the median particle size based on mass.

“Solvate” describes a molecular complex comprising the drug substance and a stoichiometric or non-stoichiometric amount of one or more pharmaceutically acceptable solvent molecules (e.g., ethanol). When the solvent is tightly bound to the drug the resulting complex will have a well-defined stoichiometry that is independent of humidity. When, however, the solvent is weakly bound, as in channel solvates and hygroscopic compounds, the solvent content will be dependent on humidity and drying conditions. In such cases, the complex will often be non-stoichiometric.

“Hydrate” describes a solvate comprising the drug substance and a stoichiometric or non-stoichiometric amount of water.

TABLE 1 lists abbreviations used throughout the specification.

TABLE 1
List of Abbreviations

| Abbreviation | Description |
|---|---|
| Å | Angstrom unit |
| ACN | acetonitrile |
| API | active pharmaceutical ingredient |
| CAP | cellulose acetate phthalate |
| CAT | cellulose acetate trimellitate |
| CEC | carboxyethylcellulose |
| CMC | carboxymethylcellulose |
| CMEC | carboxymethylethylcellulose |
| d10, d50, d90 | cumulative distribution functions in which 10%, 50% |
| | and 90% of the solids (based on volume) have |
| | diameters less than d10, d50, and d90, respectively |
| EC | ethyl cellulose |
| HDPE | high density polyethylene |
| HEC | hydroxyethyl cellulose |
| HMG-CoA | 3-hydroxy-3-methylglutaryl-coenzyme A |
| HPC | hydroxypropylcellulose |
| HPCAP | hydroxypropylcellulose acetate phthalate |
| HPCAS | hydroxypropylcellulose acetate succinate |
| HPLC | high-pressure liquid chromatography |
| HPMC | hydroxypropylmethylcellulose |
| HPMCAP | hydroxypropylmethylcellulose acetate phthalate |
| HPMCAS | hydroxypropylmethylcellulose acetate succinate |
| HPMCAT | hydroxypropylmethylcellulose acetate trimellitate |
| HPMCP | hydroxypropylmethylcellulose phthalate |
| MC | methylcellulose |
| Me | methyl |
| Mw | weight average molecular weight |
| MDSC | modulated differential scanning calorimetry |
| NVP | N-polyvinylpyrrolidone |
| PE | polyethylene |
| PEG | polyethylene glycol |
| PPG | polypropylene glycol |
| pK | pharmacokinetic |
| PVA | polyvinyl alcohol |
| PVAc | polyvinyl acetate |
| PVP | polyvinylpyrrolidone |
| PXRD | powder x-ray diffraction |
| RH | relative humidity |
| RPM | revolutions per minute |
| RT | room temperature, about 20° C. to 25° C. |
| TEC | triethyl citrate |
| TGA | thermogravimetric analysis |
| THF | tetrahydrofuran |
| TSM | twin-screw mixer |
| USP | United States Pharmacopoeia |
| VA | vinylacetate |
| v/v | volume/total volume × 100, % |
| w/v | weight (mass)/total volume × 100, % |
| w/w | weight (mass)/total weight (mass) × 100, % |

As noted above, the pharmaceutical composition comprises a solid dispersion and one or more pharmaceutically acceptable excipients. The solid dispersion includes amorphous atorvastatin or a pharmaceutically acceptable complex, salt, solvate or hydrate thereof, an optional stabilizer, and an optional plasticizer, which are dispersed in a melt-processible polymer. The active ingredient, atorvastatin, generally comprises about 10% to about 90% of the solid dispersion, often about 20% to about 60% of the solid dispersion, and more typically, about 30% to about 50% of the solid dispersion, based on weight.

Atorvastatin may be prepared using a number of methods. See, e.g., U.S. Pat. Nos. 5,003,080; 5,097,045; 5,124,482; 5,149,837; 5,216,174; 5,245,047; and 5,280,126 to D. E. Butler, C. F. Deering, A. Millar, T. N. Nanninga & B. D. Roth; U.S. Pat. Nos. 5,103,024 and 5,248,793 to A. Miller & D. E. Butler; U.S. Pat. No. 5,155,251 to D. E. Butler, T. V. Le, A. Millar & T. N. Nanninga; U.S. Pat. Nos. 5,397,792; 5,342,952; 5,298,627; 5,446,054; 5,470,981; 5,489,690; 5,489,691; and 5,510,488 to D. E. Butler, T. V. Le & T. N. Nanninga; U.S. Pat. No. 5,998,633 to T. E. Jacks & D. E. Butler; U.S. Pat. No. 6,087,511 to M. Lin & D. Schweiss; U.S. Pat. No. 6,433,213 to R. L. Bosch, R. J. McCabe, T. N. Nanninga & R. J. Stahl; and U.S. Pat. No. 6,476,235 to D. E. Butler, R. L. DeJong, J. D. Nelson, M. L. Pamment & T. L. Stuk, the complete disclosures of which are incorporated by reference.

The pharmaceutical composition may employ any pharmaceutically acceptable form of atorvastatin, including without limitation, its free form and its pharmaceutically acceptable complexes, salts, solvates, hydrates, and polymorphs. Salts include, without limitation, base addition salts, including hemi-salts. Pharmaceutically acceptable base addition salts may include nontoxic salts derived from bases, including metal cations, such as alkali or alkaline earth metal cations, as well as amines. Examples of potentially useful salts include, without limitation, aluminum, arginine, N,N′-dibenzylethylenediamine, calcium, chloroprocaine, choline, diethanolamine, diethylamine, dicyclohexylamine, ethylenediamine, glycine, lysine, magnesium, N-methylglucamine, olamine, potassium, procaine, sodium, tromethamine, zinc, and the like. For a discussion of useful base addition salts, see S. M. Berge et al., J. of Pharm. Sci., 66:1-19 (1977); see also Stahl and Wermuth, Handbook of Pharmaceutical Salts: Properties, Selection, and Use (2002).

The pharmaceutically acceptable salts of atorvastatin may be prepared by reacting its free acid with a desired base; by removing an acid- or base-labile protecting group from a suitable precursor of atorvastatin; by ring-opening a suitable cyclic precursor (lactone) using a desired base; or by converting one salt of atorvastatin to another by reaction with an appropriate acid or base or by contact with a suitable ion exchange column. All of these transformations are typically carried out in a solvent. The resulting salt may precipitate out and be collected by filtration or may be recovered by evaporation of the solvent. The degree of ionization in the resulting salt may vary from completely ionized to almost non-ionized.

Atorvastatin may exist in unsolvated and solvated forms (including hydrates) and in the form of other multi-component complexes in which the drug and at least one additional component is present in stoichiometric or non-stoichiometric amounts. Multi-component complexes (other than salts and solvates) include clathrates (drug-host inclusion complexes) and pharmaceutical co-crystals. The latter are defined as crystalline complexes of neutral molecular constituents that are bound together through non-covalent interactions. Co-crystals may be prepared by melt crystallization, by recrystallization from solvents, or by physically grinding the components together. See, e.g., O. Almarsson & M. J. Zaworotko, Chem. Comm. 1889-1896 (2004). For a general review of multi-component complexes, see J. K. Haleblian, J. Pharm. Sci. 64(8):1269-88 (1975).

Potentially useful forms of atorvastatin include all of its polymorphs, crystal habits, optical isomers, and tautomers, whether pure or not.

In addition, the pharmaceutical composition may employ prodrugs of atorvastatin. Such prodrugs may be prepared by replacing appropriate functional groups of atorvastatin with functionalities known as “pro-moieties,” as described, for example, in H. Bundgaar, Design of Prodrugs (1985). Examples of prodrugs would thus include derivatives of atorvastatin in which an ester group replaces the carboxylic acid group or an ether group replaces one or more of the hydroxyl groups.

Useful forms of atorvastatin may also include pharmaceutically acceptable isotopically labeled compounds in which one or more atoms are replaced by atoms having the same atomic number, but an atomic mass or mass number different from the atomic mass or mass number that predominates in nature. Examples of isotopes suitable for inclusion in atorvastatin include isotopes of hydrogen (²H and ³H), carbon (¹¹C, ¹³C and ¹⁴C), and nitrogen (¹³N and ¹⁵N). Isotopically labeled forms of atorvastatin may be prepared by techniques known to those skilled in the art.

As indicated above, the solid dispersion includes a melt-processible polymer that reduces or prevents conversion of amorphous atorvastatin to a crystalline form by isolating individual atorvastatin molecules or clusters of atorvastatin molecules. The fraction of atorvastatin in the solid dispersion that is amorphous may range from about 5% to about 100%, but generally ranges from about 50% to about 100%, based on weight. For the purposes of this disclosure, the drug substance is considered to be predominantly, substantially or essentially amorphous when the fraction of amorphous atorvastatin is greater than or equal to about 60%, 75% or 90%, respectively, with the balance being crystalline. In practical terms, useful solid dispersions of atorvastatin may be characterized by PXRD patterns lacking peaks that are otherwise present in PXRD patterns of crystalline atorvastatin.

The melt-processible polymer generally comprises about 10% to about 90% of the resulting solid dispersion, often about 40% to about 80% of the solid dispersion, and more typically, about 50% to about 70% of the solid dispersion, based on weight. Suitable polymers include those that reduce or prevent the conversion of amorphous atorvastatin to a crystalline form, but are otherwise inert as defined above, and exhibit aqueous solubility over at least a portion of the pH range of 1 to 8, Inclusive. Useful polymers thus include, without limitation, ionizable and nonionizable cellulosic polymers, including those having ether or ester or ether and ester substituents and copolymers thereof, including so-called “enteric” and “non-enteric” polymers; vinyl polymers and copolymers having hydroxy, alkylacyloxy, and cyclicamido substituents, including methacrylic acid copolymers and aminoalkyl methacrylate copolymers; various synthetic and naturally occurring polymeric ethers and esters of polyhydric alcohols; and mixtures thereof. In one embodiment, the melt-processible polymer is an ionic or ionizable cellulosic polymer as described herein. In one embodiment, the melt-processible polymer is a nonionizable cellulosic polymer as described herein. In one embodiment, the melt-processible polymer is a vinyl polymer as described herein. In one embodiment, the melt-processible polymer is a vinyl co-polymer as described herein. In one embodiment, the melt-processible polymer is a methacrylic acid co-polymer as described herein. In one embodiment, the melt-processible polymer is an aminoalkyl methacrylate copolymer as described herein. In one embodiment, the melt-processible polymer is a polymeric ether of a polyhydric alcohol as described herein. In one embodiment, the melt-processible polymer is a polymeric ester of a polyhydric alcohol as described herein.

Exemplary ionic cellulosic polymers include, without limitation, carboxymethylcellulose (CMC) and its sodium or calcium salts; carboxyethylcellulose (CEC); carboxymethylethylcellulose (CMEC); hydroxyethylmethylcellulose acetate phthalate; hydroxyethylmethylcellulose acetate succinate; hydroxypropylmethylcellulose phthalate (HPMCP); hydroxypropylmethylcellulose succinate; hydroxypropylcellulose acetate phthalate (HPCAP); hydroxypropylcellulose acetate succinate (HPCAS); hydroxypropylmethylcellulose acetate phthalate (HPMCAP); hydroxypropylmethylcellulose acetate succinate (HPMCAS); hydroxypropylmethylcellulose acetate trimellitate (HPMCAT); hydroxypropylcellulose butyrate phthalate; carboxymethylethylcellulose and its sodium salt; cellulose acetate phthalate (CAP); methylcellulose acetate phthalate; cellulose acetate trimellitate (CAT); cellulose acetate terephthalate; cellulose acetate isophthalate; cellulose propionate phthalate; cellulose propionate trimellitate; cellulose butyrate trimellitate; and mixtures thereof.

Exemplary nonionic cellulosic polymers include, without limitation, methylcellulose (MC); ethyl cellulose (EC); hydroxyethyl cellulose (HEC); hydroxypropylcellulose (HPC); hydroxypropylmethylcellulose (HPMC); hydroxypropylmethylcellulose acetate; hydroxyethylmethylcellulose; hydroxyethylcellulose acetate; hydroxyethylethylcellulose; and mixtures thereof.

Exemplary vinyl polymers and copolymers include, without limitation, methacrylic acid copolymers and aminoalkyl methacrylate copolymers, which are available, for example, from Rohm Pharma under the trade names EUDRAGIT® L, S, NE, RL, RS, and E. Other exemplary polymers include carboxylic acid functionalized polymethacrylates and amine-functionalized polymethacrylates; poly(vinyl acetal) diethylaminoacetate; polyvinyl alcohol (PVA); and polyvinyl alcohol/polyvinyl acetate (PVA/PVAc) copolymers; and mixtures thereof.

Additional vinyl polymers and copolymers include, without limitation homopolymers of N-polyvinyl pyrrolidone (NVP), including, for example, water-soluble polyvinylpyrrolidones (PVPs or povidones), such as KOLLIDON® 12 PF, 17 PF, 25, 30, and 90 F; water-soluble copolymers of PVP and vinylacetate (VA), such as KOLLIDON® VA64; and water-insoluble cross-linked polyvinylpyrrolidones (crospovidone), such as KOLLIDON® CL, CL-M, and SR, which are available from BASF; and mixtures thereof.

Exemplary polymeric ethers and esters of polyhydric alcohols include, without limitation, polyethylene glycol (PEG) and polypropylene glycol (PPG) homopolymers and copolymers (PEG/PPG); polyethylene/polyvinyl alcohol (PE/PVA) copolymers; dextrin; pullulan; acacia; tragacanth; sodium alginate; propylene glycol alginate; agar powder; gelatin; starch; processed starch; glucomannan; chitosan; and mixtures thereof. Other exemplary polymeric ethers include polyethylene oxides, polypropylene oxides, and polyoxyethylene-polyoxypropylene block copolymers (poloxamers) such as those available from BASF under the trade names LUTROL® F 68, F 127, and F 127-M; and mixtures thereof.

The solid dispersion may optionally include a plasticizer, which aids dispersion of the active ingredient in the melt-processible polymer. The plasticizer may comprise up to about 50% of the resulting solid dispersion, but typically comprises about 5% to about 25% of the solid dispersion, based on weight. Useful plasticizers include, without limitation, low molecular weight PEGs (Mw of about 600 or less) such as LUTROL® E 300, E 400, and E 600, which are available from BASF, and tri-block (ABA) copolymers of polyoxyethylene and polyoxypropylene, such as those available from BASF under the PLURONIC® trade name; triacetin; triethyl citrate (TEC); and mixtures thereof.

The solid dispersion of amorphous atorvastatin may also include a stabilizer, which reduces or prevents chemical degradation of atorvastatin, which may occur during preparation of the solid dispersion or during storage of the drug product. The stabilizer may comprise about 0% to about 30% of the solid dispersion, generally comprises about 1% to about 20% of the solid dispersion, and more typically comprises about 5% to about 15% of the solid dispersion, based on weight. Useful stabilizers are basic compounds, and include, without limitation, pharmaceutically acceptable salts of alkali (Group 1) metals and alkaline earth (Group 2) metals, such as sodium carbonate, dibasic sodium phosphate, potassium carbonate, calcium carbonate, calcium hydroxide, calcium sulfate, magnesium carbonate, magnesium hydroxide, magnesium oxide, magnesium silicate, magnesium aluminate, aluminum magnesium hydroxide, and mixtures thereof. For a discussion of useful stabilizers, see U.S. Pat. No. 5,686,104 to Mills et al., which is herein incorporated by reference.

As described above, the solid dispersion of amorphous atorvastatin is prepared by mixing crystalline atorvastatin or a pharmaceutically acceptable complex, salt, solvate or hydrate thereof, with one or more melt-processible polymers, an optional stabilizer, and an optional plasticizer. Mixing occurs at a temperature that is sufficiently high to soften or melt the polymer and to disperse atorvastatin and stabilizer throughout the polymeric carrier. Mixing temperatures are often high enough to melt crystalline atorvastatin in the presence of the melt-processible polymer and are typically at or above about 130° C., 140° C., 150° C., 160° C., 170° C., or 180° C. The resulting solid dispersion is subsequently allowed to cool.

A number of mechanical mixers may be used to disperse atorvastatin and the optional stabilizer in the polymeric carrier. These include twin-screw extruders (mixers), as well as high shear vertical and horizontal mixers used in melt granulation operations. Potentially useful twin-screw mixers (TSMs) include, without limitation, those available from APV/Baker, Haake, Werner Pfleiderer, and DACA. Potentially useful high shear mixers include, without limitation, those available from Niro A/S, L. B. Bohle, Machine Collette N. V. (Gral), Dierks and Sohne (Diosna), Lodige, Moritz, Processall, Roto, and Glatt.

The solid dispersion of amorphous atorvastatin may undergo further processing to prepare solid pharmaceutical compositions, including final dosage forms such as tablets, capsules, powders, and the like. For example, extruded solid dispersions may be chopped to provide granules having a median particle size of, e.g., about 0.250 mm to about 2 mm. The granules may be used directly to make drug product, or may be milled to a median particle size of, e.g., about 1 μm to about 150 μm. Useful milling equipment includes jet mills (dry), ball mills, hammer mills, and the like. The milled particles may then be combined with additional pharmaceutically acceptable excipients. The resulting mixture may be dry blended (say, in a v-cone blender) to form a drug product, which may optionally undergo further operations, such as tableting or encapsulation, coating, and the like, to prepare the final dosage form of the drug product. For a discussion of milling, dry blending, tableting, encapsulation, coating, and the like, see A. R. Gennaro (ed.), Remington: The Science and Practice of Pharmacy (20th ed., 2000); H. A. Lieberman et al. (ed.), Pharmaceutical Dosage Forms: Tablets, Vol. 1-3 (2d ed., 1990); and D. K. Parikh & C. K. Parikh, Handbook of Pharmaceutical Granulation Technology, Vol. 81 (1997), which are herein incorporated by reference.

For tablet dosage forms, depending on dose, the drug may comprise about 1% to about 80% of the dosage form, but more typically comprises about 5% to about 60% of the dosage form, based on weight. In addition to atorvastatin, the tablets may include one or more disintegrants, surfactants, glidants, lubricants, binding agents, and diluents, either alone or in combination. Examples of disintegrants include, without limitation, sodium starch glycolate; CMC, including its sodium and calcium salts; croscarmellose; crospovidone, including its sodium salt; PVP, MC; microcrystalline cellulose; one- to six-carbon alkyl-substituted HPC; starch; pregelatinized starch; sodium alginate; and mixtures thereof. The disintegrant will generally comprise about 1% to about 25% of the dosage form, or more typically, about 5% to about 20% of the dosage form, based on weight.

Tablets may optionally include surfactants, such as sodium lauryl sulfate and polysorbate 80; glidants, such as silicon dioxide and talc; and lubricants, such as magnesium stearate, calcium stearate, zinc stearate, sodium stearyl fumarate, sodium lauryl sulfate, and mixtures thereof. When present, surfactants may comprise about 0.2% to about 5% of the tablet; glidants may comprise about 0.2% to about 1% of the tablet; and lubricants may comprise about 0.25% to about 10%, or more typically, about 0.5% to about 3% of the tablet, based on weight.

As noted above, tablet formulations may include binders and diluents. Binders are generally used to impart cohesive qualities to the tablet formulation and typically comprise about 10% or more of the tablet based on weight. Examples of binders include, without limitation, microcrystalline cellulose, gelatin, sugars, polyethylene glycol, natural and synthetic gums, PVP, pregelatinized starch, HPC, and HPMC. One or more diluents may make up the balance of the tablet formulation. Examples of diluents include, without limitation, lactose monohydrate, spray-dried lactose monohydrate, anhydrous lactose, and the like; mannitol; xylitol; dextrose; sucrose; sorbitol; microcrystalline cellulose; starch; dibasic calcium phosphate dihydrate; and mixtures thereof.

### EXAMPLE 1 TO EXAMPLE 17

The following examples are intended to be illustrative and non-limiting, and represent specific embodiments of the present invention.

TABLE 2 lists ingredients of pharmaceutical formulations used in Example 1 to Example 17. Crystalline atorvastatin calcium drug substance was obtained from internal supplies. EUDRAGIT E PO, which is micronized EUDRAGIT E 100—a cationic copolymer of a diaminoethyl methacrylate and a neutral methacrylic ester—was obtained from Rohm America Inc. via Chemical Marketing Concepts. Polyethylene glycol 400 (PEG 400), calcium carbonate (CaCO₃), and triethyl citrate (TEC) were obtained from BASF, MDL Information Systems Inc., and Morflex, respectively. PVP K30, KOLLIDON SR, and KOLLIDON VA64 were obtained from BASF. HPMCAS (MG grade) was obtained from Shin-Etsu Chemical Co, Ltd.

The components of each formulation listed in TABLE 2 were premixed or blended prior to extrusion. For formulations containing at least one liquid ingredient, the solid (powder) ingredients were weighed and manually blended for about one minute using a spatula. An appropriate amount of the liquid ingredient (PEG 400 or TEC) was added to each blend of dry components. The resulting mixture was blended for about 15 minutes using a mortar and pestle and then screened through a No. 20 (0.85 mm) US standard sieve to remove any lumps that may have formed during blending. The lumps were ground with a mortar and pestle until the powdered material could pass through a No. 20 sieve. Each mixture was blended for an additional 30 minutes in an HDPE container (100 cm³) using a TURBULA shaker mixer (Glen Mills Inc.). Prior to extrusion, each blend was screened through a No. 20 sieve to ensure powder uniformity.

For formulations having no liquid components, the solid (powder) ingredients were weighed and manually blended for about one minute using a spatula. Each of the resulting mixtures was subsequently blended for an additional 30 minutes in an HDPE container (100 cm³) using a TURBULA shaker mixer. Prior to extrusion, each blend was screened through a No. 40 (0.425 mm) US standard sieve to ensure powder uniformity.

A DACA Instruments MicroCompounder twin-screw mixer (TSM) was used to prepare the pharmaceutical compositions listed in TABLE 2. The extruder employed twin conical co-rotating screws to convey, mix, and extrude small amounts of material (e.g., about 0.5 g/minute to about 1 g/minute, depending on the formulation) under controlled conditions, such as screw rotation speed, barrel temperature, and pressure. The extruder was equilibrated for 30 minutes at the desired processing temperature (170° C.) prior to processing. Each of the premixed formulations was manually fed into the feed throat of the extruder. Extrudate samples were collected after the extruder reached steady state (e.g., after about 5 minutes), were cooled at RT, and stored in desiccators for later milling and analysis. Processing temperature, screw rotational speed, and pressure were monitored throughout each run and recorded. The extruder was disassembled and cleaned between polymer (carrier) changes.

TABLE 3 lists processing parameters (TSM speed), extrudate appearance, and pH of the milled extrudate and blends prior to extrusion. The pH of each sample was determined using aqueous samples having concentrations of 0.16 mg/mL. Each sample was agitated using a wrist shaker prior to the pH measurements, which were taken at 0.5 hours, 1 hour, and 24 hours. TABLE 3 shows pH measurements after stirring for one half hour since longer stirring times did not significantly change pH.

A SPEX 6800 Freezer/Mill was used to mill the extrudate samples. Each sample was pre-cooled for 15 minutes and milled at 15 impacts per second for a minimum of 4 cycles and maximum of 6 cycles with each cycle consisting of 2 minutes of milling followed by 1 minute of cooling. Milled samples were separated based on size by using an ATM SONIC SIFTER having US standard sieve sizes of 200 (0.075 mm), 100 (0.150 mm), and 60 (0.250 mm). In some instances, the milling cycle failed to produce enough sample in the desired particle size range, so milled samples having particle size greater than 0.250 mm underwent further grinding.

Extrudate samples with particle sizes between 0.075 mm and 0.150 mm were characterized via powder x-ray diffraction (PXRD), modulated differential scanning calorimetry (MDSC), thermogravimetric analysis (TGA), pH, aqueous dissolution, and high-pressure liquid chromatography (HPLC).

Some of the milled extrudate samples stored in closed HDPE bottles at 30° C. and 60% RH and at 40° C. and 75% RH for 1 and 3 months. For the most part, the milled extrudate samples stored at 30° C. and 60% RH remained in the form of powders. Extrudate samples stored at 40° C. and 75% RH became caked except for the milled PVP K30 extrudates (Examples 10 and 11), which became hard gels. The caked samples broke easily into powder using a spatula, but the PVP K30 gels required grinding with a mortar and pestle prior to analysis. The samples stored at 40° C. and at 75% RH were analyzed for moisture content and physical and chemical stability using TGA, PXRD, and HPLC, respectively.

Powder x-ray diffractograms were obtained using a Rigaku Ultima+x-ray powder diffractometer (copper target producing 1.54 Angstrom x-rays) and scanned using a theta/2-theta goniometer. Diffractograms were obtained with the instrument operating under high sensitivity conditions (2.0 mm divergence and scatter slits; 2.0 and 0.6 mm receiving monochromator slits), a scan speed of 1 degree 2θ/minute, and a sampling interval of 0.02°2θ with the x-ray power of 40 kV/40 mA. The sample was scanned over a 2θ range of 3 to 50 degrees.

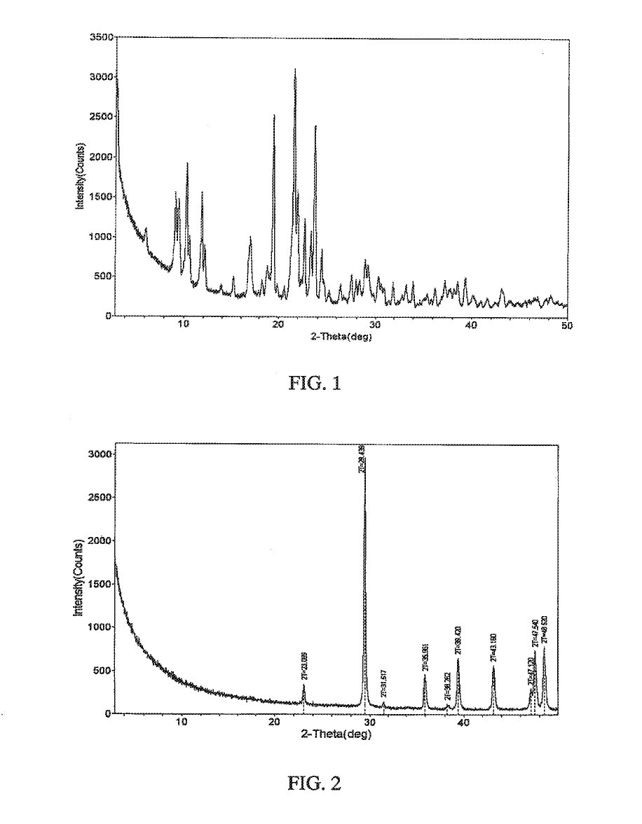

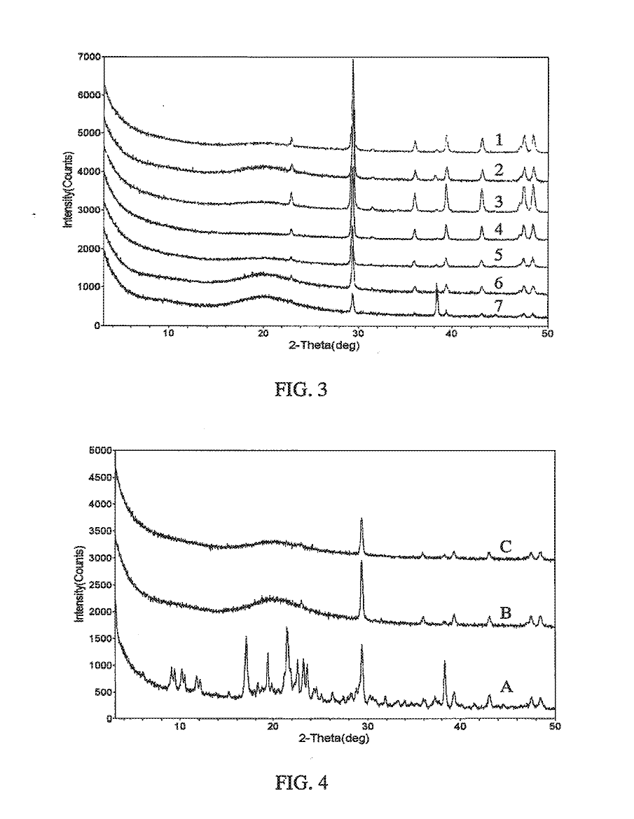

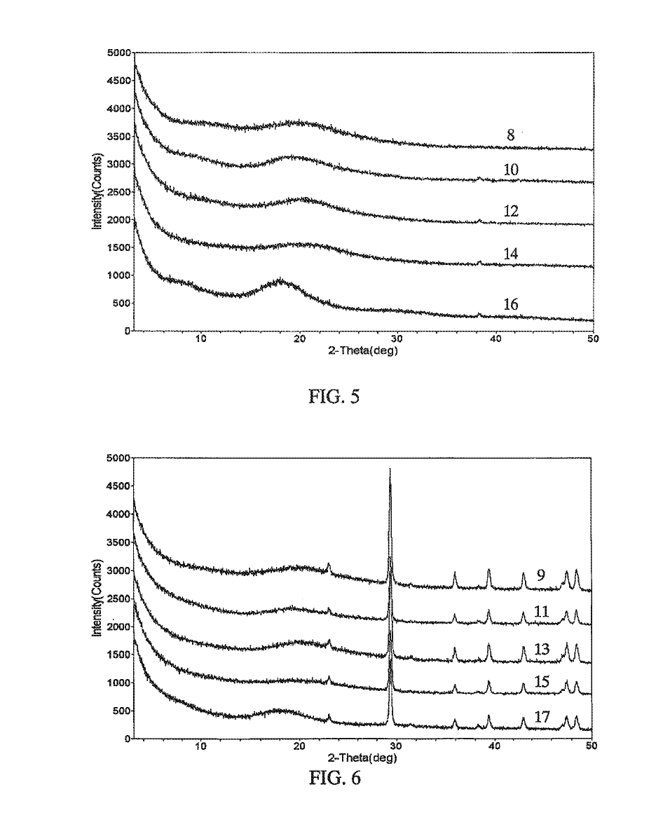

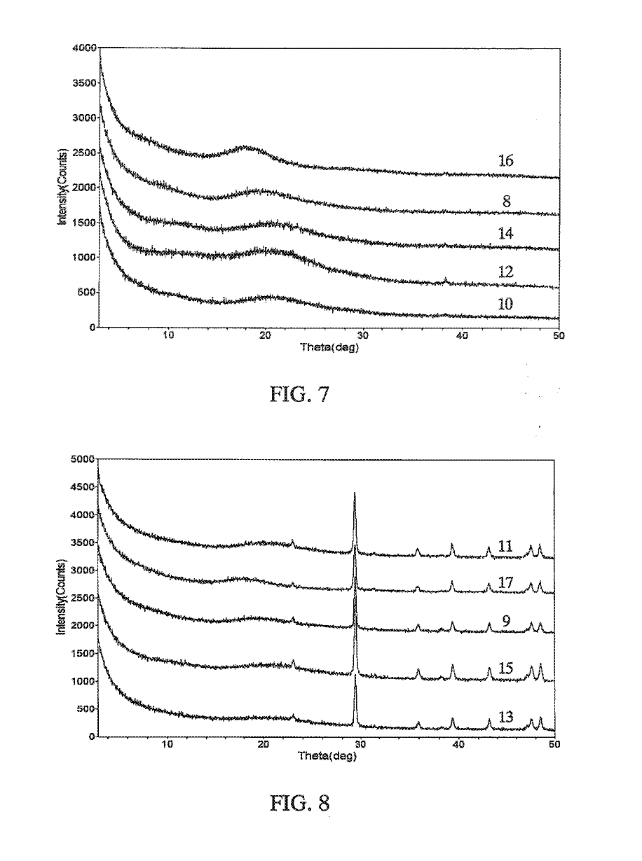

FIG. 1 and FIG. 2 show powder x-ray diffraction (PXRD) patterns for crystalline atorvastatin calcium and for calcium carbonate prior to blending. FIG. 3 shows PXRD patterns for the solid dispersions of atorvastatin in Example 1 to Example 7 following hot-melt extrusion and milling. FIG. 4 shows PXRD patterns for the formulation in Example 6 after blending, but before extrusion, following extrusion and milling, but before storage, and following extrusion, milling, and subsequent exposure to 40° C. and 75% RH for 60 hours (diffractogram A, B, and C, respectively). Similarly, FIG. 5 and FIG. 6 show PXRD patterns for the solid dispersions of atorvastatin in Example 8, 10, 12, 14, and 16 and in Example 9, 11, 13, 15, and 17, respectively, following hot-melt extrusion and milling, but before storage. FIG. 7 and FIG. 8 show PXRD patterns for the solid dispersions of atorvastatin in Example 8, 10, 12, 14, and 16 and in Example 9, 11, 13, 15, and 17, respectively, following hot-melt extrusion, milling and subsequent exposure to 40° C. and 75% RH for 3 months.

As indicated by the PXRD diffractograms shown in FIG. 3 to FIG. 8, all of the samples were amorphous immediately following extrusion and milling and after storage for 3 months at 40° C. and 75% RH. The origin of a single weak peak observed at 38° 2θ in the PXRD patterns of the extrudate is unknown. It is not due to atorvastatin since this peak was seen in the PXRD patterns of amorphous excipients such as KOLLIDON SR and HPMCAS-MG.

TABLE 4 shows dissolution of the milled extrudate in USP water as a function of time. The samples were tested using a USP Type II dissolution apparatus (37° C., 50 RPM). Three samples, each containing approximately 20 mg of active ingredient, were dissolved in USP purified water. Samples were pulled at 15, 30, 45, and 60 minutes. An additional sample was pulled after the paddle speed was increased to 100 RPM for 20 minutes. The samples were filtered through a Millipore Millex GV filter (0.22 μm porosity) and analyzed via UV/Vis spectrophotometry (244 nm wavelength, 0.5 cm path length cell) using pure atorvastatin calcium as the standard.

Assays for drug substance and degradants were carried out using an isocratic HPLC method. The method employed a Phenomenex Ultremex C18 reverse-phase, 5 μm particle size, 250×4.6 mm column using a 27:20:53 v/v/v mobile phase composition of ACN:THF:ammonium citrate (0.05 M, pH 4). Samples were analyzed using an HP 1100 HPLC system with a flow rate of 1.5 mL/minute and UV detection at 244 nm. Samples were prepared by extracting an equivalent of 10 mg active ingredient with 50:50 v/v ammonium citrate (pH 7.4):ACN to give a final concentration of 0.1 mg/mL. Samples were run for 15 minutes since no degradants were observed beyond 15 minutes in preliminary studies. The amount of degradation of atorvastatin calcium was calculated based on total area percent.

TABLE 5 shows the amount of drug substance and total degradants in the pre-extrusion blends and in the extrudate immediately following milling and after storage in closed HDPE bottles at 40° C. and 75% RH for 1 and 3 months. Assay data for extrudate samples stored for one- and three-months were adjusted to account for any changes in moisture content from the pre-storage extrudate samples.

It should be noted that, as used in this specification and the appended claims, singular articles such as “a,” “an,” and “the,” may refer to a single object or to a plurality of objects unless the context clearly indicates otherwise. Thus, for example, reference to a composition containing “a compound” may include a single compound or two or more compounds.

It is to be understood that the above description is intended to be illustrative and not restrictive. Many embodiments will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patents, patent applications and publications, are incorporated herein by reference in their entirety and for all purposes.

TABLE 2
Atorvastatin Calcium Formulations (% w/w)

| | | | | | HPMC | | | | |
|---|---|---|---|---|---|---|---|---|---|
| | | | PVP | PEG | AS- | | KOLLIDON | KOLLIDON | EUDRAGITE |
| Example | Drug | CaCO₃ | K30 | 400 | MG | TEC | SR | VA64 | PO |
| 1 | 40 | 20 | 30 | 10 | | | | | |
| 2 | 45 | 10 | 35 | 10 | | | | | |
| 3 | 35 | 30 | 25 | 10 | | | | | |
| 4 | 33.75 | 30 | 26.25 | 10 | | | | | |
| 5 | 46.67 | 10 | 33.33 | 10 | | | | | |
| 6 | 48.5 | 5 | 37 | 10 | | | | | |
| 7 | 50 | 2 | 38 | 10 | | | | | |
| 8 | 40 | | | | 50 | 10 | | | |
| 9 | 40 | 10 | | | 40 | 10 | | | |
| 10 | 40 | | 50 | 10 | | | | | |
| 11 | 40 | 10 | 40 | 10 | | | | | |
| 12 | 40 | | | | | | 60 | | |
| 13 | 40 | | | 10 | | | 50 | | |
| 14 | 40 | | | | | | | 60 | |
| 15 | 40 | | | 10 | | | | 50 | |
| 16 | 40 | | | | | | | | 60 |
| 17 | 40 | | | 10 | | | | | 50 |

TABLE 3
TSM Speed, Extrudate Appearance and pH

| | Speed | | Blend | Extrudate |
|---|---|---|---|---|
| Example | RPM | Extrudate Appearance | pH | pH |
| 1 | 75 | Lt cream, opaque, brittle | 9.60 | 9.61 |
| 2 | 75 | Lt cream, opaque, brittle | 9.67 | 9.74 |
| 3 | 75 | Lt cream, opaque, brittle | 9.75 | 9.69 |
| 4 | 75 | Lt cream, opaque, brittle | 9.69 | 9.67 |
| 5 | 75 | Lt cream, opaque, brittle | 9.70 | 9.69 |
| 6 | 75 | Lt cream, opaque, brittle | 9.28 | 9.19 |
| 7 | 75 | Lt cream, opaque, brittle | 8.48 | 8.10 |
| 8 | 75 | Lt brown, transparent, brittle | 7.10 | 6.51 |
| 9 | 75 | Lt brown, opaque, brittle | 9.22 | 8.35 |
| 10 | 75 | Lt yellow, foams, transparent, | 6.42 | 6.35 |
| | | brittle | | |
| 11 | 75 | Off-white to yellow, opaque, | 9.55 | 9.45 |
| | | brittle | | |
| 12 | 75 | Off-white, opaque, brittle | 7.48 | 6.71 |
| 13 | 75 | White, foams, brittle | 9.41 | 8.36 |
| 14 | 75 | Lt yellow, transparent, brittle | 7.22 | 6.63 |
| 15 | 75 | White to off-white, opaque, | 9.51 | 9.19 |
| | | brittle | | |
| 16 | 50 | Lt yellow, transparent, brittle | 7.07 | 6.80 |
| 17 | 50 | Very Lt yellow, opaque, brittle | 9.51 | 8.45 |

TABLE 4
Amount of Extrudate Dissolved in USP Water (n = 3)

| | % (w/w) @ time = | | | | |
|---|---|---|---|---|---|
| Example | 15 min | 30 min | 45 min | 60 min | 70 min |
| 8 | 60.1 | 62.4 | 63.9 | 66 | 68.7 |
| 9 | 72.7 | 90.4 | 99 | 100.8 | 100.7 |
| 10 | 85 | 90.2 | 90.5 | 90.9 | 91.8 |
| 11 | 92.1 | 99.1 | 98.4 | 99.3 | 99.1 |
| 12 | 45.5 | 61.8 | 67.8 | 72.6 | 82 |
| 13 | 39.5 | 51.3 | 58.1 | 62.4 | 72.1 |
| 14 | 82.7 | 90 | 95.1 | 97.7 | 99.4 |
| 15 | 88 | 97 | 101.3 | 101.7 | 101.9 |
| 16 | 3 | 3.2 | 3.7 | 4.3 | 6.1 |
| 17 | 3 | 3.6 | 3.8 | 4.2 | 4.9 |
| Bulk drug | 70.2 | 89.8 | 95.9 | 97.9 | 99.9 |

TABLE 5
Amount (w/w) of Drug Substance and Total Degradants
Before (Blend) and After Extrusion (Extrudate)

| | | | Initial | | 1 Month | | 3 Month | |
|---|---|---|---|---|---|---|---|---|
| | Blend | | Extrudate | | Extrudate | | Extrudate | |
| | | % Total | % | % Total | | % Total | | % Total |
| Example | % Drug | Degradants | Drug | Degradants | % Drug | Degradants | % Drug | Degradants |
| 1 | 97.26 | 0.11 | 98.27 | 1.05 | | | | |
| 2 | 100.58 | 0.11 | 96.46 | 0.94 | | | | |
| 3 | 101.40 | 0.12 | 96.94 | 1.18 | | | | |
| 4 | 98.55 | 0.12 | 98.26 | 1.15 | | | | |
| 5 | 98.41 | 0.11 | 93.91 | 0.83 | | | | |
| 6 | 96.03 | 0.13 | 99.06 | 1.45 | | | | |
| 7 | 98.81 | 0.13 | 99.26 | 1.54 | | | | |
| 8 | 99.43 | 0.67 | 55.78 | 41.07 | 66.77 | 29.33 | 54.60 | 38.31 |
| 9 | 97.53 | 0.39 | 62.43 | 36.83 | 72.13 | 25.02 | 62.48 | 30.69 |
| 10 | 97.70 | 0.12 | 97.44 | 1.36 | 88.58 | 2.18 | 80.50 | 7.02 |
| 11 | 96.20 | 0.12 | 101.16 | 1.01 | 90.30 | 1.54 | 82.74 | 7.70 |
| 12 | 101.05 | 0.12 | 100.18 | 0.99 | 97.2 | 1.63 | 94.12 | 3.62 |
| 13 | 99.32 | 0.12 | 100.17 | 0.90 | 97.15 | 1.3 | 94.72 | 3.17 |
| 14 | 100.93 | 0.12 | 98.81 | 2.30 | 94.2 | 1.85 | 89.63 | 4.52 |
| 15 | 100.10 | 0.12 | 96.76 | 2.36 | 94.8 | 1.87 | 93.03 | 4.39 |
| 16 | 100.85 | 0.12 | 99.36 | 1.85 | 98.48 | 1.13 | 98.25 | 3.05 |
| 17 | 100.95 | 0.12 | 98.42 | 2.11 | 99.10 | 1.40 | 99.61 | 2.90 |

1. A solid pharmaceutical composition comprising a solid dispersion of amorphous atorvastatin and one or more optional pharmaceutically acceptable excipients, the solid dispersion comprising:
amorphous atorvastatin or a pharmaceutically acceptable complex, salt, solvate or hydrate thereof; and
a melt-processible polymer.

2. The solid pharmaceutical composition of claim 1, wherein the melt-processible polymer is a cellulosic polymer, a vinyl polymer, a vinyl co-polymer, a methacrylic acid copolymer, an aminoalkyl methacrylate copolymer, a polymeric ether of a polyhydric alcohol, or a polymeric ester of a polyhydric alcohol, either alone or in combination.

3. The solid pharmaceutical composition of claim 1, wherein the melt-processible polymer is an aminoalkyl methacrylate copolymer.

4. The solid pharmaceutical composition of claim 1, further comprising a plasticizer.

5. The solid pharmaceutical composition of claim 4, wherein the plasticizer is triethyl citrate or a polyethylene glycol having a weight average molecular weight of about 600 or less.

6. The solid pharmaceutical composition of claim 1, wherein the amorphous atorvastatin comprises from about 10% to about 90% of the solid dispersion based on weight.

7. The solid pharmaceutical composition of claim 1, wherein the amorphous atorvastatin comprises from about 20% to about 60% of the solid dispersion based on weight.

8. The solid pharmaceutical composition of claim 1, wherein the amorphous atorvastatin comprises from about 30% to about 50% of the solid dispersion based on weight.

9. The solid pharmaceutical composition of claim 1, wherein the pharmaceutical composition is a final dosage form.

10. The solid pharmaceutical composition of claim 9, wherein the final dosage form is a tablet, a capsule, or a powder.

11. A solid pharmaceutical composition comprising a solid dispersion of amorphous atorvastatin and one or more optional pharmaceutically acceptable excipients, the solid dispersion comprising:
amorphous atorvastatin or a pharmaceutically acceptable complex, salt, solvate or hydrate thereof;
a melt-processible polymer; and
a stabilizer for reducing chemical degradation of the amorphous atorvastatin.

12. The solid pharmaceutical composition of claim 11, wherein the stabilizer is a pharmaceutically acceptable salt of an alkaline metal or alkaline earth metal.

13. A method of making a solid pharmaceutical composition, the method comprising:
mixing crystalline atorvastatin or a pharmaceutically acceptable complex, salt, solvate or hydrate thereof, with a melt-processible polymer at a temperature sufficiently high to soften or melt the melt-processible polymer and to melt or dissolve the crystalline atorvastatin in the melt-processible polymer, thereby forming a dispersion of amorphous atorvastatin; and
allowing the dispersion to cool.

14. The method of claim 13, wherein mixing occurs at a temperature sufficiently high to melt crystalline atorvastatin in the presence of the melt-processible polymer.

15. The method of claim 13, wherein mixing occurs at a temperature at or above 130° C., 140° C., 150° C., 160° C., 170° C., or 180° C.

16. The method of claim 13, wherein the melt-processible polymer is polyvinylpyrrolidone, polyvinylpyrrolidone/vinylacetate copolymer, a methacrylic acid copolymer, an aminoalkyl methacrylate copolymer, a polymeric ether of a polyhydric alcohol, or a polymeric ester of a polyhydric alcohol, either alone or in combination.

17. The method of claim 13, further comprising mixing atorvastatin with a plasticizer.

18. The method of claim 13, further comprising mixing atorvastatin with a plasticizer, wherein the plasticizer is triethyl citrate or a polyethylene glycol having a Mw of about 600 or less.

19. The method of claim 13, further comprising mixing atorvastatin with a stabilizer, the stabilizer adapted to reduce chemical degradation of atorvastatin.

20. The method of claim 13, further comprising mixing atorvastatin with a stabilizer, wherein the stabilizer is a pharmaceutically acceptable salt of an alkaline metal or alkaline earth metal.

21. The method of claim 13, further comprising mixing crystalline atorvastatin and the melt processible polymer in a twin-screw mixer.
